(19) United States
(12) Patent Application Publication
(10) Pub. No.: US 2024/0050391 A1
(43) Pub. Date: Feb. 15, 2024

(54) COMPOSITIONS AND METHODS FOR NEUROPROTECTION IN NEONATAL HYPOXIC-ISCHEMIC ENCEPHALOPATHY

(71) Applicant: The Trustees of Columbia University in The City of New York, New York, NY (US)

(72) Inventors: Richard J. DECKELBAUM, Hastings on Hudson, NY (US); Hylde ZIRPOLI, Long Island City, NY (US); Soren Weis DAHL, New York, NY (US); Vadim S. TEN, Staten Island, NY (US)

(21) Appl. No.: 18/265,500

(22) PCT Filed: Dec. 7, 2021
(86) PCT No.: PCT/US21/62102

§ 371 Date: Jun. 6, 2023

Related U.S. Application Data
(60) Provisional application No. 63/122,198, filed on Dec. 7, 2020.

(51) Int. Cl. A61K 31/202; A61K 9/107; A61P 25/00; A61K 47/24

(52) U.S. Cl. CPC A61K 31/202; A61K 9/107; A61P 25/00; A61K 47/24

(57) ABSTRACT

In various aspects and embodiments of the disclosure, the invention provides pharmaceutical compositions and methods for protecting against brain injury associated with Hypoxic-Ischemic Encephalopathy (HIE). The compositions and methods employ omega-3 fatty acid (n-3 FA) diglyceride (DG) and/or triglyceride (TG) emulsions, which may be used alone or in combination with therapeutic Hypothermia (HT).

### BACKGROUND

Hypoxic-ischemic encephalopathy (HIE) is a serious occurrence in neonates that frequently results in death or significant long-term developmental and neurologic disabilities, such as cerebral palsy. HIE is caused by a range of conditions that result in oxygen deprivation to the newborn brain, including before, during, or after birth. Currently, therapeutic hypothermia (HT) is the only established treatment for HIE. HT involves cooling the whole body or the brain of the newborn to around 33-34° C. for 72 hours. Preclinical studies and small scale clinical trials have shown that HT can diminish the degree of neural damage, reduce the rate of mortality, and improve neurofunctional recovery.

However, HT is only successful in reducing long-term neurological impairments in a minority of HIE patients (about 1 in 8), and its use is generally limited to tertiary care facilities; therefore, HT can require the transport of the newborn, delaying the treatment at a critical time. HT generally must be started within 6 hours from the ischemic event.

Accordingly, compositions and methods to provide neuroprotection for neonates experiencing or at risk of HIE are urgently needed.

### SUMMARY OF THE DISCLOSURE

In various aspects and embodiments, the invention provides pharmaceutical compositions and methods for protecting against brain injury associated with Hypoxic-Ischemic Encephalopathy (HIE). The compositions and methods employ omega-3 fatty acid (n-3 FA) diglycerides (DG) and/or triglycerides (TG), which can be formulated into emulsions. In accordance with the various aspects and embodiments, this disclosure provides compositions and methods for preventing or reducing brain damage in the baby caused by oxygen deprivation and/or limited blood flow during the prenatal, intrapartum or postnatal period.

In various embodiments, the baby is prenatal or intrapartum and is at risk of HIE, and the n-3 FA DG or TG emulsions are administered to the pregnant mother intravenously. For example, in some embodiments, where an ischemic event potentially triggering HIE is anticipated or detected, the n-3 FA DG or TG emulsions are administered intravenously to the mother, for example, upon detection of preeclampsia or other condition of pregnancy associated with HIE risk. In some embodiments, the emulsion is administered during labor, including prolonged labor or where umbilical cord compression is determined to place the newborn at risk for HIE, or when blood flow to the placenta is impaired such as in abruption placenta.

In other embodiments, the subject for administration is a neonate (i.e., a newborn) where HIE is suspected. The n-3 FA DG or TG emulsions are administered to the newborn within about twelve hours of delivery, or within about ten hours of delivery, or within about eight hours of delivery, or within about six hours of delivery, or within about four hours of delivery, or within about two hours of delivery. For delivery to a neonate, the emulsions may be administered via a nasogastric (NG) tube or may be administered intravenously.

In various embodiments, the neonate is further treated with HT, either before, during, or after treatment with the n-3 FA TG or DG emulsions. HT should generally be initiated as soon as possible, such as within about six hours of delivery. However, in some embodiments, the HT is initiated within about twelve hours of delivery, or within about ten hours of delivery, or within about eight hours of delivery. In some embodiments, administration with TG or DG emulsions shortly after birth in may expand the window during which HT provides benefit for avoiding brain injury and long term and/or lifelong complications of HIE.

In accordance with the invention, the fatty acids of the DGs or TGs are predominately n-3 FAs. In some embodiments, the n-3 FAs are long chain n-3 FAs, including one or more of docosahexaenoic acid (DHA), eicosapentaenoic acid (EPA), and docosapentaenoic acid (DPA). In some embodiments, the n-3 FAs comprise DHA and EPA. EPA and DHA may optionally be present at a ratio of about 4:1 to about 1:4 (e.g., about 1:1). In still other embodiments, the FAs may further comprise arachidonic acid (ARA) or medium chain fatty acids (MCFA). In some embodiments, the DGs or TGs (or emulsions) further comprise one or more specialized pro-resolving mediators (SPMs), which are oxygenated metabolites derived mainly from AA, EPA, DPA, and DHA. They include lipoxins, (neuro)protectins, resolvins, and maresins and can have potent anti-apoptotic, anti-inflammatory and immunoregulatory effects at concentrations in the nanomolar to picomolar range.

In various embodiments, the emulsions comprise about 10% to about 30% TG oil and/or DG oil by weight of the total composition together with one or more emulsifiers. For example, emulsions may comprise about 15% to about 30% of the DG oil by weight of the total composition, or about 15% to about 25% of the DG oil by weight (e.g., about 20% DG oil), with one or more emulsifiers.

In various embodiments, the subject (e.g., such as a neonatal subject experiencing HIE or at risk of HIE) receives one or more bolus injections or infusions of the emulsion, or via a nasogastric (NG) tube. In some embodiments, the subject may receive at least two and up to six administrations of the emulsions. In various embodiments, the emulsions are administered over the course of one day to about one week, and may be administered one or more times daily (e.g., about daily).

As an exemplary embodiment, this disclosure provides a method for protecting against brain injury associated with HIE in a neonate, where the method comprises administering to a pregnant mother carrying a child at risk of HIE, either prenatal or intrapartum, an intravenous injection of n-3 FA DG or TG emulsions. After delivery, the newborn may optionally be further treated with a HT regimen and/or DG or TG emulsion therapy. In some embodiments, the DG or TG emulsions are administered intravenously to the mother before or during labor, once HIE is anticipated or is at significant risk.

In other exemplary embodiments, the method for neuroprotection comprises administering to a neonatal subject in need, n-3 FA DG emulsions within about twelve hours of delivery, or within about ten hours of delivery, or within about eight or six hours of delivery (or less) and treating the neonatal subject with a HT regimen. Emulsions may be administered before, during, and/or after the HT regimen. For example, at least a first dose of the emulsion may be administered within about six hours of delivery, or within about four hours of delivery, within about two hours of delivery, thereby providing neuroprotection during the time the newborn is being transported to initiate HT. The emulsions are administered via a nasogastric (NG) tube or intravenously. In accordance with these embodiments, HT can be initiated later than generally desired, and still provide unexpected therapeutic benefit. In still other embodiments, HT is initiated first, and n-3 FA TG or DG emulsion administered subsequently.

In other aspects, this disclosure provides a pharmaceutical composition, which can be used for the various methods described herein. In various embodiments, the pharmaceutical compositions comprise an effective amount of n-3 FA DG emulsions, where the DG comprise at least about 50% EPA and DHA, and comprise from 1% to about 40% arachidonic acid (ARA) and/or medium chain fatty acids (MCFAs) (with respect to the total FA content by weight), and one or more emulsifiers.

In some embodiments, the emulsions comprise about 10% to about 30% diglyceride oil by weight. In some embodiments, the emulsions comprise about 15% to about 25% diglyceride oil by weight. The compositions will further comprise one or more emulsifiers to obtain the desired physical characteristics. In various embodiments, emulsifiers can include one or more of phospholipid emulsifiers, phosphoglyceride emulsifiers, and medium and/or long chain fatty acid emulsifiers. Phosphoglyceride emulsifiers may be selected from phosphatidylcholine, phosphatidylethanolamine, phosphatidylinositol, phosphatidylserine, and phosphatidic acid. Medium chain or long chain FAs as co-emulsifier may be selected from lauric acid, myristic acid, palmitic acid, stearic acid, oleic acid and linolenic acid. An exemplary co-emulsifier is sodium oleate.

In various embodiments, the composition optionally comprises one or more polyols, such as glycerol. For example, the composition may comprise glycerol at from about 2% to about 10% by weight of the composition. In some embodiments, the composition further comprises one or more anti-oxidants, such as one or more of α-tocopherol, β-tocopherol, γ-tocopherol, and an ascorbyl ester. In some embodiments, the composition further comprises a metal chelating agent, which is optionally EDTA or EGTA.

Further aspects and embodiments of the disclosure will now be described by the following detailed description.

### DESCRIPTION OF THE FIGURES

FIG. 1A through FIG. 1C. Effect of hypothermia (HT) and tri-DHA on infarct volume: Infarct volume (FIG. 1A) and representative TTC stained cerebral sections (FIG. 1B) in mice treated with normothermia (NT)+saline (n=21), HT+saline (n=20), tri-DHA (n=21) or HT+tri-DHA (n=11) beginning immediately (0 h) after hypoxic-ischemic (HI) injury (% infarct volume: NT+saline-33.2±4.8, HT+saline-14.9±4.8, 0 h tri-DHA-14.9±4.5, 0 h HT+tri-DHA-15.0±7.0); representative Nissl-stained cerebral coronal sections from mice treated with NT+saline, HT+saline or HT+tri-DHA beginning immediately (0 h) after HI injury (FIG. 1C). Values are mean±SEM. *p<0.05, compared to NT+saline group.

FIG. 2A through FIG. 2D. Therapeutic windows of hypothermia (HT) and tri-DHA: Infarct volume (FIG. 2A) and representative TTC stained cerebral sections FIG. 2(B) in mice treated with normothermia (NT)+saline (n=22), HT+saline (n=22) or HT+tri-DHA (n=21) beginning at 2 h after hypoxic-ischemic (HI) injury (% infarct volume: NT+saline-31.4±4.1, 2 h delayed HT+saline-18.8±4.6, 2 h delayed HT+tri-DHA-12.7±4.0); Infarct volume (FIG. 2C) and representative TTC stained cerebral sections (FIG. 2D) in mice treated with normothermia (NT)+saline (n=19), HT+saline (n=23) or HT+tri-DHA (n=10) beginning at 4 h after hypoxic-ischemic (HI) injury (% infarct volume: NT+saline-30.7±5.0, 4 h delayed HT+saline-31.3±5.6, 4 h delayed HT+tri-DHA-41.3±5.5). Values are mean±SEM. *p<0.05, **p<0.01, compared to NT+saline group.

FIG. 3. Infarct volumes in neonatal mice (10-day old) subjected to hypoxic-ischemic (HI) injury and treated with saline as vehicle, n-3 triglyceride (TG), n-3 diglyceride (DG) emulsions. n=15-17. Values are means with SD. Dose=0.375 mg/g bw.

FIG. 4. Infarct volumes in neonatal mice (10-day old) subjected to hypoxic-ischemic (HI) injury and treated with saline as vehicle or neuroprotectin from DHA (PD1) at the concentrations of 10 or 20 ng per animal and compared to neuroprotectin from DPA (PD1 n-3 DPA) at 10 ng per animal. n=15-17. Values are means with SD.

### DETAILED DESCRIPTION OF EMBODIMENTS

In various aspects and embodiments, the invention provides pharmaceutical compositions and methods for protecting against brain injury associated with Hypoxic-Ischemic Encephalopathy (HIE). The compositions and methods employ omega-3 fatty acid (n-3 FA) diglyceride (DG) and/or triglyceride (TG) oils which are formulated into emulsions. In accordance with the various aspects and embodiments, this disclosure provides compositions and methods for preventing or reducing brain damage caused by oxygen deprivation and/or limited blood flow during the prenatal, intrapartum or postnatal period. Such conditions are often described clinically as (neonatal) Hypoxic Ischemic Encephalopathy (HIE), which includes neonatal encephalopathy birth asphyxia, intrapartum asphyxia and perinatal asphyxia. HIE can result in various types of tissue damage, and can result in long-term disabilities such as cerebral palsy, cognitive disability, epilepsy, hearing and visual impairments, among others.

Causes of HIE during pregnancy (i.e., which place the newborn at risk for HIE) include preeclampsia, eclampsia, gestational diabetes, and infection. Other conditions during pregnancy or labor which place the newborn at risk for HIE include placental abruption, placenta previa, placental insufficiency, uterine rupture, and umbilical cord complications. Umbilical cord complications include compression of the umbilical cord, which can occur, for example, during prolonged labor. Changes in fetal heart rate or uterine tachysystole before delivery can be predictors of HIE. During the neonatal period, premature infants are particularly at risk of HIE, since the lungs may be underdeveloped. Generally, conditions that signify risk of HIE in neonates include respiratory distress, jaundice, and neonatal hypoglycemia. Signs of HIE include breathing problems, feeding problems, lack of reflexes, seizures, and low level of consciousness.

HIE or risk of HIE in the newborn can be diagnosed or evaluated using Apgar score. The five criteria assessed in the Apgar score are: (1) Appearance (i.e., skin color/tone); (2) Pulse; (3) Grimace (i.e., response to stimuli); (4) Activity (i.e., muscle tone/activity level; and (5) Respiration. Generally, the five criteria are each scored as 0, 1 or 2 (two being the best), and the total score is calculated by adding the five values. Scores of 0-3 are considered critical, especially in babies born at or near term. Scores of 4-6, especially after 5 minutes, are considered below normal and may indicate risk of HIE and that the medical intervention may be required. Scores of 7+ are considered normal.

Alternatively or in addition, risk of HIE can be assessed by sampling the cord blood immediately after birth. Blood pH and base deficit (BD) are known means for assessing whether an ischemic event has occurred. Other predictors of HIE include known serum markers of brain injury including brain-specific creatine kinase (CK-BB), protein S-100B, neuron specific endolase, and other inflammatory markers such as interleukins. Nagdyman N., et al. Early biochemical indicators of hypoxic-ischemic encephalopathy after birth asphyxia. Pediatr Res. 2001 April; 49(4):502-6. Other imaging and neurophysiological tests can also be employed to determine early risk assessment as well as provide long-term prognostication, and these include Amplitude-integrated EEG, T1-weighted and T2-weighted MRI, Thalamic Magnetic Resonance Spectroscopy (MRS), and Deep gray matter lactate/N-acetyl aspartate (Lac/NAA) peak/area ratio. Pang R., et al., Proton Magnetic Resonance Spectroscopy Lactate/N-Acetylaspartate within 48 h Predicts Cell Death Following Varied Neuroprotective Interventions in a Piglet Model of Hypoxia—Ischemia With and Without Inflammation-Sensitization. Front Neurol. 2020; 11: 883.

The present disclosure in some embodiments provides pharmaceutical compositions and methods using stable omega-3 DG or TG oil-in-water emulsions for acute therapy. The DG or TG emulsions in various embodiments contain about 50% to about 100% omega-3 fatty acids (with respect to total weight of fatty acids), including DHA and EPA, and optionally DPA. In some embodiments, total fatty acids further include ARA or MCFAs. The DG emulsions are suitable for parenteral delivery, such as intravenous (i.v.) route, and the physical properties of DG and TG emulsions facilitate rapid delivery of the omega-3 fatty acids to the damaged brain tissue. In some embodiments, the DG or TG emulsions are generally about 10% to about 30% by weight of the composition. That is, there is about 10 to about 30 g of DG or TG oil per 100 mL emulsion. Exemplary compositions of emulsions are described elsewhere herein.

Administration of the emulsions (e.g., n-3 FA DG emulsions) as described herein can provide synergistic protection with therapeutic Hypothermia (HT), or can act as an alternative to HT. HT is a currently accepted treatment for HIE. HT is a procedure used to slow down the injury process associated with HIE. HT involves cooling the baby to about 33.5 to 34.5° C. for about 72 hours, ideally initiated within about six hours of birth or the oxygen-depriving event. HT can employ a cooling cap for selective brain cooling, or a cooling blanket for whole body cooling. During the treatment, vital signs like respiration, oxygenation, heart rate, and brain wave activity are monitored. Following HT, the body is re-warmed slowly (over at least four hours), at a rate of about 0.5° C. per hour.

This disclosure investigates whether co-treatment with HT and triglyceride-DHA (tri-DHA) would achieve synergic effects in protecting the brain from HI injury in a neonatal mouse model. It was observed that HT, beginning immediately after HI injury, reduced brain infarct volume similarly to tri-DHA treatment (˜50%). These results indicate that HT offers similar degrees of neuroprotection against HI injury compared to tri-DHA treatment. HT can only be provided in tertiary care centers, requires intense monitoring and can have adverse effects. In contrast, tri-DHA treatment may be advantageous in providing a feasible and effective alternative strategy in neonates immediately after HI injury, and will not require transport of the newborn from the hospital to a specialized facility. Thus, in some embodiments, the subject does not undergo HT therapy, and the TG or DG emulsions are provided as an alternative therapy. In other embodiments, TG or DG emulsion is provided to the subject prior to HT, for example, while preparing for HT or prior to or during transport of the subject to the specialized facility capable of performing HT. DG emulsions provide a surprisingly more effective treatment than TG, and given their high level of effectiveness, provide improved benefits compared to HT, or synergistic benefits along with HT. In other embodiments, the TG or DG emulsions are first administered during or after HT.

While some mechanistic pathways of HIE are similar to adult stroke, the pathobiology of HIE injury is believed to be distinct from that of adult stroke. In fact, therapies designed to ameliorate brain injury in adults could worsen outcomes in neonates, possibly by accentuating apoptosis.

For example, since myelination is still occurring in the neonatal brain and the water content of the neonatal brain is greater than that of the mature brain, injury has a different appearance and time-course in the neonatal brain than in the adult brain. Cell death mechanisms have been shown to be different in the developing brain compared to that in the adult (Zhu C, et al. The influence of age on apoptotic and other mechanisms of cell death after cerebral hypoxia-ischemia. Cell Death Differ. 2005, 12, 162-176). The mechanisms of mitochondrial permeabilization are age-dependent and while Cyclophilin D is critical in the adult brain, Bax-related mechanisms dominate in the immature brain (Wang X, et al. Developmental shift of cyclophilin D contribution to hypoxic-ischemic brain injury. J. Neurosci. 2009, 29, 2588-2596). In comparison with the adult, the immature brain has a high expression of many pro-apoptotic proteins (including Bax, caspase-2, -3, -8 and -9), and apoptosis occurs following mitochondrial outer membrane permeabilization (MOMP). Pathways dependent on apoptotic-inducer factor (AIF) (Zhu C, et al. Involvement of apoptosis-inducing factor in neuronal death after hypoxia-ischemia in the neonatal rat brain. J. Neurochem. 2003, 86, 306-317) and caspases seem to be more strongly activated in the immature brain than in the adult brain (Hu B R, et al. Involvement of caspase-3 in cell death after hypoxia-ischemia declines during brain maturation. J. Cereb. Blood Flow Metab. 2000, 20, 1294-1300, and mitochondrial permeabilization has been proposed to mark the point of no return in hypoxic—ischemic injury of the immature brain (Hagberg H. Mitochondrial impairment in the developing brain after hypoxia-ischemia, J. Bioenerg. Biomembr. 2004, 36, 369-373). In the immature brain, cyclophilin D gene deficiency aggravates rather than lessens hypoxic—ischemic injury compared to adult mice (Wang et al., 2009; Schinzel A C, et al. Cyclophilin D is a component of mitochondrial permeability transition and mediates neuronal cell death after focal cerebral ischemia. Proc. Natl. Acad. Sci. U.S.A. 2005, 102, 12005-12010). Instead, BAX-inhibitory peptides (Wang X. et al. Neuroprotective effect of Bax-inhibiting peptide on neonatal brain injury. 2010, Stroke 41, 2050-2055) and BAX deficiency (Gibson M E, et al. BAX contributes to apoptotic-like death following neonatal hypoxia-ischemia: evidence for distinct apoptosis pathways. Mol. Med. 2001, 7, 644-655) substantially protect the immature brain in neonatal mice.

Further, stroke triggers a robust inflammatory response in both adult and neonatal brain. Experimental evidence from adult models show that brain injury rapidly activates microglia and lead to increased phagocytic activity and altered production of cytokines and reactive oxygen metabolites (Hanisch U K. Microglia as a source and target of cytokines. 2002, Glia 40, 140-155), features that are also well documented in neonatal hypoxic-ischemic (HI) injury (Hedtjärn M, et al. Inflammatory gene profiling in the developing mouse brain after hypoxia-ischemia. J. Cereb. Blood Flow Metab. 2004, 24, 1333-1351. However, compared to the adult, microglial activation in neonates is much more rapid following ischemic injury. In the adult brain there is also a considerable contribution of infiltrating peripheral immune cells to the brain after stroke injury (Iadecola C and Anrather J. The immunology of stroke: from mechanisms to translation. 2011, Nat. Med. 17, 796-808). In contrast, little infiltration of peripheral cells is seen acutely after neonatal stroke (Denker S P, et al. Macrophages are comprised of resident brain microglia not infiltrating peripheral monocytes acutely after neonatal stroke. J. Neurochem. 2007, 100, 893-904). Thus, these findings suggest differences in neonatal and adult central nervous system immune responses to injury.

Further still, blood brain barrier is markedly more intact in neonatal than in adult animals after acute ischemic brain injury, in part because of the differential expression of the basal lamina and tight junction proteins and neutrophil behavior (Fernández-López D, et al. Blood-brain barrier permeability is increased after acute adult stroke but not neonatal stroke in the rat. J Neurosci. 2012 Jul. 11; 32(28):9588-600).

Thus, in various embodiments, the subject is prenatal or intrapartum and is at risk of HIE (that is, HIE is anticipated), and the n-3 FA DG or TG emulsion is administered to the pregnant mother, for example, intravenously. For example, the n-3 FA DG or TG emulsions may be administered intravenously to the mother upon detection of preeclampsia or other condition of pregnancy associated with HIE risk (as already described). In some embodiments, the emulsion is administered before or during labor, including prolonged labor or where umbilical cord compression is determined to place the new born at risk for HIE. As described in more detail herein, the emulsions may be administered intravenously as one or more bolus doses or by continuous infusion, or as a combination of one or more bolus loading doses followed by infusion of one or more additional doses. For example, dose(s) administered i.v. can be administered as a bolus (over the course of 0 to 60 min) or via infusion (over the course of 60 min to 24 hrs) or as a combination of one or more bolus loading doses followed by infusion of one or more additional doses. Alternatively, the emulsion can be administered by NG tube.

In other embodiments, the subject is a neonate (i.e., a newborn), and HIE is suspected. In various embodiments, the neonate is preterm or term. The n-3 FA DG or TG emulsions are administered to the newborn within about twelve hours of delivery, or within about ten hours of delivery, or within about eight hours of delivery, or within about six hours of delivery, or within about four hours of delivery, or within about two hours of delivery. For delivery to a neonate, the emulsions may be administered via a NG tube or may be administered intravenously, either as a bolus dose and/or as a continuous infusion.

In various embodiments, the neonate is further treated with HT, either before, during, or after n-3 FA DG or TG administration. The HT should generally be initiated as soon as possible, such as within about six hours of delivery, or within about four hours of delivery. However, in some embodiments, the HT is initiated within about twelve hours of delivery, or within about ten hours of delivery, or within about eight hours of delivery. In these embodiments, administration with the DG or TG emulsions in particular may expand the window during which HT provides benefit for avoiding brain injury and long term and/or lifelong complications of HIE. Thus, in such embodiments, the HT is initiated after about six hours of delivery, or after about eight hours of delivery, or after about ten hours of delivery, or after about twelve hours of delivery, which may be due to a delay in detecting HIE or in transporting the newborn from the hospital to a tertiary care facility that is equipped for HT.

In various embodiments, after treatment with the DG or TG emulsions and/or HT, the neonatal patient may be further treated with parenteral nutrition involving omega-3 FAs. These can include infusion of about 1 g/kg/day of TG emulsions, and which can be prepared from about 10% fish oil (e.g., about 25% to about 60% EPA and DHA). Such emulsion is commercially available under the name Omegaven. Omegaven is an intravenous lipid emulsion that provides calories and fatty acids with anti-inflammatory effects for pediatric patients with parenteral nutrition-associated cholestasis, or PNAC. Such parenteral nutrition can be provided for several weeks to months, such as for two to about twenty weeks, or for about four to about sixteen weeks.

In various embodiments, the present invention delivers n-3 FAs to cells as stable DG or TG emulsions. The term “n-3 FAs” means a polyunsaturated FA where one of the carbon-carbon double bonds is between the third and fourth carbon atoms from the distal end of the hydrocarbon chain. Examples of n-3 FAs include α-linolenic acid (18:3n-3; α-ALA; Δ^{3,6,9}), eicosapentaenoic acid (20:5n-3; EPA; Δ^{5,8,11,14,17}), docosahexaenoic acid (22:6n-3; DHA; Δ^{4, 7,10,13,16,19}) and docosapentaenoic acid (22:5n-3; DPA; Δ^{7,10,13,16,19}). n-3 FAs having at least 20 carbon atoms are referred to as “long chain n-3 FAs”. Sources of n-3 FAs may be from any suitable source such as from fish oils, algae oils and other oils or may be synthesized.

DGs are composed of two FAs esterified to the trihydric alcohol glycerol. An exemplary method for synthesis of DG molecules is through lipase-catalyzed glycerolysis (i.e., transesterification) with n-3 long chain FAs. An exemplary process for preparing the DG oil is described in WO 2019/234057, which is hereby incorporated by reference in its entirety. As used herein, a DG oil refers to an oil in which at least about 75% of the glycerides are diacylglycerides. However, in some embodiments, at least about 80%, or at least about 90% (e.g., about 100%) of the glycerides are diacylglycerides. Likewise, when TG emulsions are employed, a TG oil refers to an oil in which at least about 75% of the glycerides are triacylglycerides. However, in some embodiments, at least about 80%, or at least about 90% (e.g., about 100%) of the glycerides are triacylglycerides.

In accordance with the invention, the FAs of the DGs or TGs may be predominately n-3 FAs. In various embodiments, the DG or TG comprise at least about 50% n-3 FAs (by weight of the total fatty acids), or at least about 75% n-3 FAs, or at least about 90% n-3 FAs, or about 100% n-3 FAs. In some embodiments, the n-3 FAs are long chain n-3 FAs, including one or more of DHA, EPA, and DPA. Interestingly, neuroprotection D1 derived from DPA are neuroprotective (as measured by decreasing infarct size after hypoxic-ischemic brain injury) at lower concentrations than neuroprotection D1 derived from DHA (FIG. 4).

In various embodiments, the n-3 FAs comprise DHA. In some embodiments, the n-3 FAs are at least about 50% DHA, or at least about 60% DHA, or at least about 75% DHA, or at least about 90% DHA. In some embodiments, the n-3 FAs comprise EPA. For example, the n-3 FAs may be at least about 50% EPA, or at least about 60% EPA, or at least about 75% EPA, or at least about 90% EPA. In some embodiments, the n-3 FAs comprise DHA and EPA. For example, the FAs may comprise at least about 50% EPA and DHA, or at least about 60% EPA and DHA, or at least about 70% EPA and DHA, or at least about 80% EPA and DHA, or at least about 90% EPA and DHA. EPA and DHA may optionally be present at a ratio of about 4:1 to about 1:4, such as about 3:1 to about 1:3, or about 2:1 to about 1:2 (e.g., about 1:1). In various embodiments, the DG molecules are one or more of 1,2-DGs or 1,3-DGs. In some embodiments, the DGs are predominately 1,3-DGs.

In still other embodiments, the fatty acids further comprise ARA, such as about 1% to about 40% ARA (by weight of total fatty acids in the glycerides), or in some embodiments, about 1% to about 30%, or about 5% to about 25%, or about 5% to about 20%, or about 10% to about 20% ARA. ARA is a key n-6 fatty acid important for brain growth in infants. In these or other embodiments, the emulsions further comprise MCFAs, either as free FAs or esterified as TGs and/or DGs. TGs rich in MCFAs enhance efficiency of omega-3 fatty acids to cell membranes. In some embodiments, the MCFA are present in the range of about 1% to about 20% by weight of the fatty acids in the TG and/or DG emulsions. In some embodiments, the composition comprises medium chain triglycerides (MCT) as described in U.S. Pat. No. 9,675,572, which is hereby incorporated by reference in its entirety.

In some embodiments, the DG or TG (or emulsions thereof) comprise one or more SPMs. The SPMs are oxygenated metabolites derived mainly from AA, EPA, DPA, and DHA. They include lipoxins, (neuro)protectins, resolvins, and maresins and can have potent anti-apoptotic, anti-inflammatory and immunoregulatory effects at concentrations in the nanomolar to picomolar range. SPMs are produced by dioxygen-dependent oxidation from their n-3 FA and n-6 FA precursors. SPMs include certain AA-derived lipoxins (LXA4 and LXB4), EPA-derived E-series resolvins (RvE1-3), DHA-derived D-series resolvins (RvD1-6), protectins/neuroprotectins (PD1/NPD1 and PDX), maresins (MaR1 and MaR2), and DPA-derived 13-series resolvins (RvT1-4). SPMs can act as immunoresolvents.

In some embodiments, the DG or TG (or emulsion thereof) comprises at least one SPM derived from EPA, such as Resolvin E1, Resolvin E2, and Resolvin E3. In some embodiments, the DG or TG (or emulsion thereof) comprises at least one SPM derived from DHA, such as Resolvin D1, Resolvin D2, Resolvin D3, Resolvin D4, Resolvin D5, Resolvin D6, or a stereoisomer thereof. In some embodiments, the DG or TG (or emulsion thereof) comprises at least one SPM derived from DPA such as Resolvin T1, Resolvin T2, Resolvin T3, Resolvin T4, Resolvin 1 n-3 DPA, Resolvin 2 n-3 DPA, Resolvin 5 n-3 DPA, Protectin 1 n-3 DPA, Protectin 2 n-3 DPA, Maresin 1 n-3 DPA, Maresin 2 n-3 DPA, and Maresin 3 n-3 DPA. In some embodiments, the DG of TG (or emulsion thereof) comprises at least one bioactive lipid derived from arachidonic acid (AA) such as Lipoxin A4, Lipoxin B4, or a stereoisomer thereof.

In various embodiments, the emulsions comprise about 10% to about 30% TG oil and/or DG oil by weight of the total composition. For example, the emulsions may comprise about 15% to about 25% (e.g., about 15%, about 20% or about 25%) DG oil by weight of the total composition. Other components of the emulsions (e.g., emulsifiers) are described elsewhere herein.

In various embodiments, the subject (e.g., such as a neonatal subject experiencing HIE or at risk of HIE) receives a single bolus injection or infusion of the emulsion or via NG. For example, the subject may receive at least two and up to six (e.g., from 2 to 4) bolus injections or infusions of the emulsion. In various embodiments, the bolus injections or infusions are administered no more frequently than about once every 12 hours. However, in various embodiments administrations can be spaced by interims independently selected from about one hour, about 2 hours, about 3 hours, about six hours, about 12 hours, or about 24 hours. In various embodiments, the emulsions are administered over the course of about one day to about one week, and may be administered about daily. In various embodiments, the bolus administration is from about 0.05 g to about 5 g of the emulsified DG or TG per kg body weight, or about 0.5 to about 5 g per kg body weight. For example, the bolus administration may be about 2 g to about 4 g of the emulsified DG or TG per kg body weight.

As an exemplary embodiment, this disclosure provides a method for protecting against brain injury associated with Hypoxic-Ischemic Encephalopathy (HIE) in a neonate, where the method comprises administering to a pregnant mother carrying a child at risk of HIE, either prenatal or intrapartum, an intravenous injection of n-3 FA DG or TG emulsions. After delivery, the newborn may optionally be further treated with a HT regimen and/or DG or TG therapy. In some embodiments, the emulsion is administered intravenously to the mother before or during labor, once HIE is anticipated or is at significant risk. Administration and the composition of the emulsions are as described elsewhere herein. The newborn may be a preterm or term neonate.

In some embodiments, after birth, a further n-3 DG or TG emulsion dose is administered to the newborn within about twelve or within about ten hours of delivery (i.e., birth), or within about six or within about eight hours of delivery, or within about four hours of delivery, within about two hours of delivery. The timing of HT after delivery, which is generally as soon as possible, is as already described. In various embodiments, the neonate receives a single bolus injection or infusion of the emulsion (or by NG route). However, in some embodiments the neonate receives at least two and up to six (e.g., 2 to 4) bolus injections or infusions of the emulsion. These are generally administered over the course of about one week, and may be administered about daily by intravenous and/or NG routes.

In other exemplary embodiments, the method for neuroprotection comprises administering to a neonatal subject in need, n-3 DG or TG emulsions within about ten hours of delivery, and treating the neonatal subject with a HT regimen. N-3 DG or TG emulsion is first administered before, during, or after HT. For example, at least a first dose of the emulsion is administered within about six hours of delivery, or within about four hours of delivery, within about two hours of delivery, thereby providing neuroprotection during the time the newborn is being transported to a specialized facility to initiate HT. The DG or TG emulsion is administered via a nasogastric (NG) tube or intravenously, either as a bolus and/or as a continuous infusion as already described.

In accordance with these embodiments, HT can be initiated later than generally desired, and still provide unexpected therapeutic benefit. For example, in some embodiments, a dose of DG or TG emulsion is administered within about four hours of delivery, and HT is initiated within about 10 hours of delivery. In some embodiments, a dose of DG or TG emulsion is administered within about four hours of delivery, and HT is initiated within about eight hours of delivery or within about six hours of delivery.

In still other embodiments, a dose of DG or TG emulsion is administered within about two hours of delivery (or within about one hour of delivery), and HT is initiated after about six hours of delivery, or after about eight hours of delivery, or after about ten hours of delivery, or after about twelve hours of delivery. In various embodiments, the subject receives a single bolus injection or infusion of the emulsion, but in other embodiments, the subject receives at least two and up to six (e.g., two to four) bolus injections or infusions of the emulsion. These may be generally administered over the course of about one day to one week, such as about daily.

In other aspects, this disclosure provides a pharmaceutical composition, which can be used for the various methods described herein. In various embodiments, the pharmaceutical compositions comprise an effective amount of n-3 DG or TGs emulsions, where the DGs or TGs comprise at least about 50% EPA and DHA and from 1% to about 40% or from 1% to about 30%, or from 1% to about 20%, or from 1% to about 10% ARA (with respect to the total FA content by weight), and one or more emulsifiers. In some embodiments, the DGs or TGs comprise about 5% to about 25%, or about 5% to about 20%, or about 10% to about 20% ARA by weight of the FAs). In some embodiments, the FAs comprise at least about 60% EPA and DHA, or at least about 70% EPA and DHA, or at least about 80% EPA and DHA. In some embodiments, the ratio of EPA:DHA is about 4:1 to about 1:4, or about 3:1 to about 1:3, or about 2:1 to about 1:2, and optionally about 1:1. Generally, the composition comprises at least about 75% DG or TG, with respect to the total of monoacylglycerides, diacylglycerides, and triacylglycerides.

In some embodiments, the fatty acids further comprise DPA and/or MCFAs, either as free FAs or esterified as DGs or TGs. In various embodiments, the MCFA(s) is about 1% to about 20% of the fatty acids in the diglyceride emulsions (with respect to total FA by weight). In some embodiments, the emulsions comprise about 10% to about 30% DG or TG oil by weight. In some embodiments, the emulsions comprise from about 15% to about 25% (e.g., about 15%, about 20%, or about 25%) DG or TG oil by weight.

In accordance with the compositions and methods described herein, the compositions are stable emulsions that can be stored in stable form for use in the emergency setting. The emulsions described herein are substantially stable for at least six months, or at least one year, at 4° C. In various embodiments, the emulsions are stable for more than about one year (e.g., about 18 months or about 2 years) at 4° C., or in some embodiments at room temperature (i.e., about 22° C.). The compositions are suitable for parenteral delivery routes, such as intravenous or intra-arterial delivery, or via NG route. Further, in some embodiments the physical properties of the emulsions facilitate delivery of the n-3 FAs to, and/or uptake by, brain tissue.

Emulsions are inherently unstable and, thus, do not form spontaneously. Energy input through shaking, stirring, homogenizing, for example, is needed to form an emulsion. Over time, emulsions tend to revert to the stable state of the phases comprising the emulsion. However, nanoemulsions can be kinetically stable.

If the size and dispersion of droplets of an emulsion do not substantially or significantly change over a desired time frame (such as at least about six months), the emulsion is said to be stable. That is, emulsion stability refers to the ability of an emulsion to resist changes in its properties over time. Instability in emulsions can be observed as, for example, flocculation, creaming/sedimentation, and coalescence. Flocculation occurs when there is an attractive force between the droplets, so they form flocs. Coalescence occurs when droplets combine to form a larger droplet, so that the average droplet size increases over time. Emulsions can also undergo creaming, where the droplets rise to the top of the emulsion under the influence of buoyancy, for example. Sedimentation is the opposite phenomenon of creaming and normally observed in water-in-oil emulsions. Sedimentation happens when the dispersed phase is denser than the continuous phase and the gravitational forces pull the denser globules towards the bottom of the emulsion. Similar to creaming, sedimentation follows Stokes' law. Other measures that inform on stability include the no increases in free FA amounts or oxidative products in the emulsion or no decreases in tocopherol concentrations over time (e.g., 6 to 24 months).

An emulsifier is a substance that stabilizes an emulsion by increasing its kinetic stability. Emulsifiers include surface active agents, or surfactants. Surfactants can increase the kinetic stability of an emulsion so that the size of the droplets does not change significantly with time. The stability of an emulsion can be evaluated in terms of zeta potential, which indicates the repulsion between droplets or particles. Emulsifiers are compounds that typically have a polar or hydrophilic (i.e. water-soluble) part and a non-polar (i.e. hydrophobic or lipophilic) part. Detergents are a type of emulsifier, and will interact physically with both oil and water, thus stabilizing the interface between the oil and water droplets in suspension.

In various embodiments, the emulsions have a mean particle size of 200 nm or less and a zeta potential of about −40 mV or more negative than about −40 mV. In some embodiments, the mean particle size of the emulsions is about 180 nm or less, or about 150 nm or less, or about 120 nm or less, or about 100 nm or less, or about 90 nm or less, or about 80 nm or less. In some embodiments, the mean particle size is about 120 nm, or about 110 nm, or about 100 nm, and with a polydispersion index of less than about 0.3 or less than about 0.2. In various embodiments, the zeta potential of the emulsions is at least as negative as about −45 mV, or at least as negative as about −50 mV, or at least as negative as about −55 mV, or at least as negative as about −60 mV. The emulsions in accordance with these embodiments are stable, meaning these parameters are maintained for at least six months, or in some embodiments, at least one year. In accordance with this disclosure, stability is determined with storage at about 5° C. or room temperature (i.e., about 25° C.).

The stable emulsions are suitable for i.v. administration for example, to rapidly deliver n-3 FAs to the brain. The lipid phase will generally be from about 10% to about 50% by weight of the composition. In some embodiments, the lipid phase is from about 10% to about 40% by weight of the composition, or from about 15% to about 40%, or from about 15% to about 30%, or from about 15% to about 25%, or from about 20% to about 25% by weight of the composition. For example, the lipid phase may be about 20% of the composition by weight, or about 25% of the composition by weight, or about 30% of the composition by weight. In some embodiments, at least about 10% by weight of the lipid phase is DG or TG, or at least about 15% by weight of the lipid phase is DG or TG, or at least about 20% by weight of the lipid phase is DG or TG, or about 30% by weight of the lipid phase is DG or TG, or at least about 40% by weight of the lipid phase is DG or TG, or at least about 50% of the lipid phase is DG or TG. In some embodiments, the DGs themselves provide emulsifying properties, and thus less emulsifiers are needed, as compared to TG emulsions. For example, in some embodiments, the emulsifier is about 0.5 to about 2% by weight of the composition, such as about 1.2% by weight of the composition, or about 1%, or about 0.8%, or about 0.6% by weight of the composition.

Polydispersion index (PDI) is a measure of particle size distribution within a given sample. The numerical value of PDI ranges from 0.0 (for a sample with perfectly uniform particle size distribution) to 1.0 (for a highly polydisperse sample with multiple particle size populations). In lipid-based carriers, such as emulsions, a PDI of 0.3 is desired, indicating a sufficiently homogenous particle size distribution. In some embodiments, the PDI of the emulsions is less than about 0.3, such as about 0.2 or less, or about 0.1 or less.

The compositions will comprise one or more emulsifiers to obtain the desired physical characteristics. In various embodiments, emulsifiers can include one or more of phospholipid emulsifiers, phosphoglyceride emulsifiers, and medium and/or long chain fatty acid emulsifiers. In various embodiments, the composition comprises from about 0.6% to about 10% by weight of emulsifiers, and optionally about 0.6% to about 7% by weight of emulsifiers, and optionally from about 0.6 to about 5% of emulsifiers by weight, and optionally from about 0.6% to about 3% by weight.

In some embodiments, emulsions comprise one or more phospholipid emulsifiers and/or one or more phosphoglyceride emulsifiers. Phosphoglyceride emulsifiers may be selected from phosphatidylcholine, phosphatidylethanolamine, phosphatidylinositol, phosphatidylserine, and phosphatidic acid. In some embodiments, the composition comprises a phosphatidylcholine emulsifier.

In various embodiments, the ratio of phospholipid and/or phosphoglyceride emulsifier to DG or TG is from about 1:4 to about 1:12, and in some embodiments is no more than about 1:10. In some embodiments, the emulsifier comprises at least about 70% phosphatidylcholine, or comprises at least about 80% phosphatidylcholine. For example, the emulsifier (with any co-emulsifier) may contain from about 60% to about 80% phosphatidylcholine.

The composition may further comprise one or more of medium chain or long chain FAs as co-emulsifier. For example, the composition may comprise a long chain FA, optionally selected from a C16 to C24 FA, and which is optionally a C18 FA. In some embodiments, the co-emulsifier comprises a saturated FA, optionally selected from lauric acid, myristic acid, palmitic acid, and stearic acid. In some embodiments, the co-emulsifier comprises an unsaturated FA, optionally selected from oleic acid or linolenic acid. The co-emulsifier may be added as an alkali metal salt, which optionally comprises sodium oleate. In exemplary embodiments, the co-emulsifier is present at about 0.01% to 5% of the total weight of the composition. For example, the co-emulsifier may be present from about 0.01 to 2% of the total weight of the composition, or from about 0.01% to about 1% of the total weight of the composition, or from about 0.01% to about 0.05% by weight of the composition.

In various embodiments, the composition is approximately isotonic with human blood, and optionally comprises one or more polyols, such as glycerol, sorbitol, xylitol, and/or glucose. For example, the composition may comprise glycerol at from about 2% to about 10% by weight of the composition, or from about 2% to about 7% by weight of the composition.

In some embodiments, the composition comprises one or more antioxidants, such as one or more of α-tocopherol, β-tocopherol, γ-tocopherol, and an ascorbyl ester. In exemplary embodiments, the antioxidants comprise α-tocopherol and/or ascorbyl ester, which is optionally ascorbyl palmitate.

In some embodiments, the composition comprises a metal chelating agent, which is optionally EDTA or EGTA. For example, emulsions may contain from about 0.1 mM to about 5 mM, or about 0.1 mM to about 1 mM EDTA or EGTA. For example, in some embodiments, the emulsions contain about 0.25 mM EDTA.

In various embodiments, stable emulsions can be prepared according to a process comprising: (1) preparing a mixture of water, glycerol, and EDTA having a temperature of from about 50° C. to about 80° C. (e.g., about 60° C.); (2) add phosphatidylcholine emulsifier (e.g., at least about 75% PC, which may be from egg yolk lecithin), co-emulsifier (e.g., sodium oleate), and DG or TG oil; (3) homogenize at a temperature of from about 50° C. to about 80° C. (e.g., about 60° C.); (4) process through a microfluidizer. The pressure applied during this process could range from 300 to 2000 bar, and in some embodiments, from about 500 to about 1000 bar, such as from about 600 to about 900 bar. For example, the mixture can be processed through the microfluidizer at about 800-bar pressure at about 60° C. The emulsions can be processed for a length of time and under conditions required to meet the target particle size.

In various embodiments, the pH of the composition is from about 6 to about 9, and optionally from about 6.5 to about 8.5, and optionally from about 7 to about 8.

In various embodiments, the composition has a volume of about 500 mL or less, or a volume of about 300 mL or less, or a volume of about 50 mL or less, or a volume of about 25 mL or less. In various embodiments, the composition is contained in a pre-filled syringe, optionally having a volume for injection of from about 1 mL to about 50 mL.

The composition is generally delivered parenterally, such as intravenously or intra-arterially. In some embodiments, the composition is administered intranasally, allowing for rapid delivery to the brain.

As used herein, the term “about” means±10% of the associated numerical value.

Exemplary embodiments will now be described by the following Examples.

### EXAMPLES

### Example 1: Acute Injection of Omega-3 Triglyceride Emulsion Provides Similar Neuroprotection as Therapeutic Hypothermia Against Brain Hypoxic-Ischemic Injury in a Neonatal Mouse Model

Hypoxic-ischemic (HI) brain injury is a serious occurrence that frequently results in death or significant long-term neurologic disability in both neonates and adults (1-3). Currently, therapeutic hypothermia (HT) is the only established treatment for neonates with HI encephalopathy (4). Preclinical studies and small scale clinical trials have shown that HT can diminish the degree of neural damage, reduces the rate of mortality and improve neurofunctional recovery (7,8).

The major molecular mechanisms affected by HT include decreased free-radical production, limitation of blood—brain barrier disruption, decreased excitatory amino acid release and attenuation of cell mediated inflammatory responses to cerebral ischemia (9,10). Additionally, HT induces inhibition of neuronal apoptosis through both mitochondrial based intrinsic pathways and receptor mediated extrinsic pathways (11). However, HT remains a complex medical approach, as it requires intense monitoring and is available only in tertiary care centers (12). Pilot studies on HT in stroke have shown that adult patients have less tolerance to cooling than neonates and HT may also induce unfavorable systemic effects, such as shivering, immune suppression and pneumonia (13,14). Combining HT with other treatment methods may aim in reducing the adverse effects from HT as well as reaching multiple molecular targets in the setting of HI insult to obtain an increase in therapeutic time windows and an enhanced repair in long-term recovery (15).

As one of the major omega-3 polyunsaturated FAs in the brain, DHA is essential for development and function of the brain (16). DHA has been shown to reduce inflammation, excitotoxicity and to prevent brain volume loss in different animal models of HI injury (17-19). This example investigates whether co-treatment with HT and triglyceride DHA (“tri-DHA”) emulsions would achieve synergic effects in protecting the brain from HI injury. In this example, tri-DHA is a triglyceride oil where >98% of TG fatty acids are DHA. We confirmed the neuroprotective efficacy of HT against HI injury in the neonatal model previously described (20,22). These results show that tri-DHA provides similar degrees of neuroprotection as that of HT and combining HT with tri-DHA emulsion does not offer additional therapeutic benefit in HI injury.

### Materials and Methods

DHA TG oil was purchased from Nu-Chek Prep, Inc. (Elysian, MN). Egg yolk phosphatidylcholine was obtained from Avanti Polar-Lipids, Inc. (Alabaster, AL). Radiolabeled [³H]-cholesteryl hexadecyl ether was purchased from PerkinElmer (Boston, MA) ([³H]CEt) (NET 85900).

Tri-DHA emulsions (10 g by TG weight/100 mL emulsion) were made with DHA TG oil and egg yolk phospholipids (PL) by sonication as previously detailed (20). The emulsions were analyzed for the amount of TG and PL using commercial kits (Wako Chemicals USA, Inc., Richmond, VA). The TG:PL mass ratio was 5.0±1.0, similar to VLDL-sized particles. To prepare radiolabeled emulsions, [³H]CEt was added to the TG-PL mixture before sonication (22).

Three-day-old C57BL/6J neonatal mice were purchased from Jackson Laboratories (Bar Harbor) with their birth mother. Both male and female mice were used for the experiments. We used the Rice-Vannuci method of mild HI brain injury in 10-day old (p10) mice, as previously described (20). Briefly, HI brain injury was induced by permanent ligation of the right common carotid artery. After 1.5 h of recovery, mice were exposed to hypoxic insult (humidified 8% O₂/92% N₂, Tech Air Inc., NY) for 15 min, at 37±0.3° C.

Immediately after HI injury, pups were kept for 4 h in temperature controlled chambers with either HT or normothermia (NT), reaching rectal temperatures of 31-32° C. or 37° C., respectively (23). We observed that pups placed in circulating air chambers set at 27° C. maintained target rectal temperature 31-32° C. For the NT group, pups were placed in chambers set at 32° C., based on the protocol from our previous studies (20,21). HI brain injury in neonatal mice is associated with an endogenous drop in body core temperature (23). Hence, pups kept at 37° C. during hypoxia are not subjected to hypothermia. As the core temperature in neonatal rodents could be affected by distance from the dam (24), the pups were kept separately from the dam during the 4 h HT or NT treatment period. Sequential temperature measurements were obtained immediately after hypoxia (0 h) followed by 1, 2, 3 and 4 h during HT (probe type: RET-4; Physitemp Instruments, Clifton, NJ). Tri-DHA treatment (0.375 g tri-DHA/kg bw, intraperitoneal (i.p.), two injections, 1 h apart) was based on prior protocol (20,21).

To investigate whether combined treatment of HT with tri-DHA emulsion enhances neuroprotection in HI damage, animals subjected to HT were administered with tri-DHA emulsion (0.375 g tri-DHA/kg bw, 2 injections, i.p.) at the beginning of HT and at 1 h after initiation of HT. NT or HT control animals received saline injections. Following 4 h NT, pups in the control group were returned to the dam. Pups in the HT group underwent slow rewarming by increasing the chamber temperature at a rate of 0.1-0.2° C. per minute till the pups reached a rectal temperature of 37° C., and were then returned to the dam.

Using radiolabeled tri-DHA emulsion, we determined whether HT affects the absorption and distribution of emulsion particles after i.p. injection. Naïve neonatal mice injected with radiolabeled tri-DHA emulsion (0.375 g tri-DHA/kg bw, i.p., single injection) were immediately subjected to 4 h of either HT (n=3) or NT (n=7). The use of a single bolus injection to study emulsion distribution was based on previously established protocols (22,25). Animals were sacrificed after 4 h of HT or NT and radioactivity in peritoneal fluid, blood, organs and tissues assessed by measuring the levels of [³H]CEt.

Tissues and organs were homogenized using a Polytron Tissue Disruptor (Omni TH, Kenneswa, GA) and the radioactivity measured by liquid scintillation spectrometry (26). The samples were suspended in scintillation fluid (Ultima Gold scintillation fluid, PerkinElmer, Boston, MA), mixed and 3H dpm assayed in a PerkinElmer Tri-Carb liquid scintillation spectrometer 5110 TR. Tissue uptake was expressed as percent of total recovered dose/organ for all the organs analyzed.

We determined the therapeutic window of HT after HI injury in mice: (1) 2 h delayed HT—pups placed with dam for 2 h after HI and then subjected to HT; (2) 4 h delayed HT —pups placed with dam for 4 h after HI and then subjected to HT. To investigate whether combined treatment of HT with tri-DHA emulsion prolongs the therapeutic window in HI injury, animals subjected to HT (2 h or 4 h delayed after HI) were administered with tri-DHA emulsion (0.375 g tri-DHA/kg bw, 2 injections, i.p.) at the beginning of HT and at 1 h after initiation of HT. NT or HT control animals received saline injections. After the treatment period, pups in NT or HT groups were returned to the dam as described above.

At 24 h after HI insult, the animals were sacrificed and brains were harvested. Coronal slices of 1 mm were cut by using a brain slicer matrix. Slices were immersed in a PBS solution containing 2% triphenyltetrazolium chloride (TTC) at 37° C. for 25 min. TTC is taken up into living mitochondria, which converts it to a red color. Unstained areas that appeared white were defined as infarct regions whereas viable regions appeared red. Using Adobe Photoshop and NIH Image J imaging applications, planar areas of infarction on serial sections were summed to obtain the volume (mm3) of infarcted tissue. Infarct areas were expressed as % of the total area of the ipsilateral hemisphere (21). In a separate cohort of mice treated with HT or HT plus tri-DHA immediately after HI, brain atrophy at 7 days after HI injury was detected by Nissl staining. The entire brain was sectioned every 200 μm and the thickness of each coronal slice was 50 μm. Sections were then incubated in a solution of 0.1% cresyl violet (Sigma-Aldrich, St. Louis, MO, USA) for 7 min. After a quick rinse in H₂O, slides were differentiated in 70% (v/v) ethanol with a few drops of acetic acid, followed by dehydration in graded ethanol and two changes of xylene. The sections were then mounted with Fisher Chemical™ Permount™ Mounting Media. Results were expressed as % ipsilateral hemisphere volume (residual tissue brain) compared to contralateral hemisphere (27).

Values are mean±SEM. One-way ANOVA followed by post hoc Newman-Keuls multiple comparison test was applied to evaluate differences among the groups.

### Results

There was no mortality in animals subjected to NT or HT protocols. Table 1 summarizes results of sequential temperature measurements in HT animals. Radiolabeled experiments showed that at 4 h after i.p. injection, ˜96% of the injected emulsion exited the peritoneal cavity in both NT and HT mice. Further, no significant differences were observed in the organ distribution of tri-DHA emulsion particles in NT vs. HT mice. The highest uptake of emulsion particles was in the liver (44-47% of recovered dose of radiolabeled emulsion), followed by muscle (20-23%) and heart (8-9%) in both NT and HT mice. The lowest uptake of emulsion particles was in the brain (<0.3% of recovered dose) in both NT and HT animals.

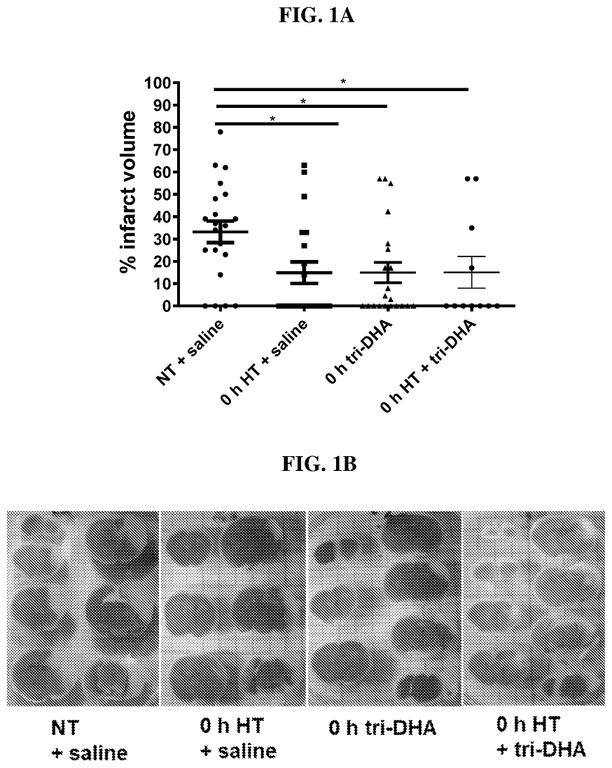

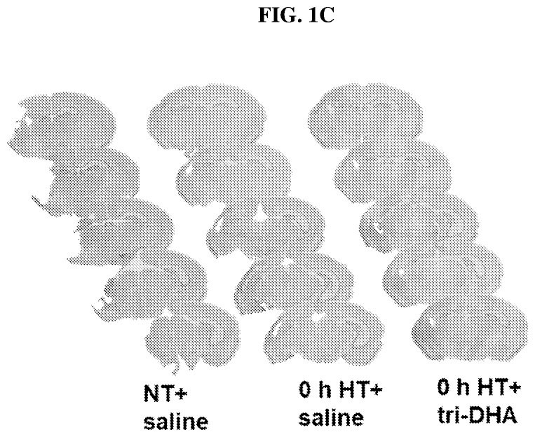

We evaluated neuroprotective effects of HT plus tri-DHA treatment beginning immediately after HI injury. HT or tri-DHA showed significant reduction (˜50%) in brain infarct volumes compared to saline treated NT animals (FIGS. 1A and 1B). Combination of treatments with HT and tri-DHA immediately after HI injury did not provide any additional benefits compared to HT treatment alone.

Neuroprotection by HT plus tri-DHA administration beginning immediately after HI injury was maintained at 7 days after ischemic insult. Nissl staining demonstrated greater preservation of the ipsilateral hemisphere in HT or HT plus tri-DHA treated mice compared to the control group. However, the combination did not offer any therapeutic advantage compared to HT treatment alone. Representative Nissl stained sections are shown in FIG. 1C.

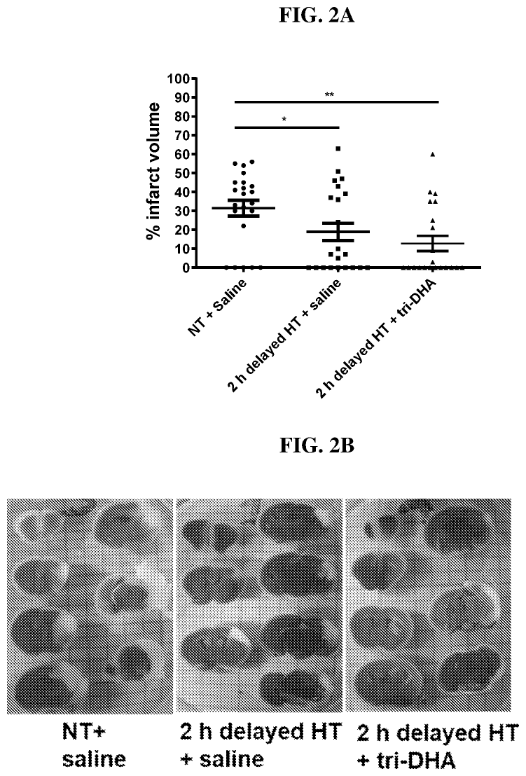

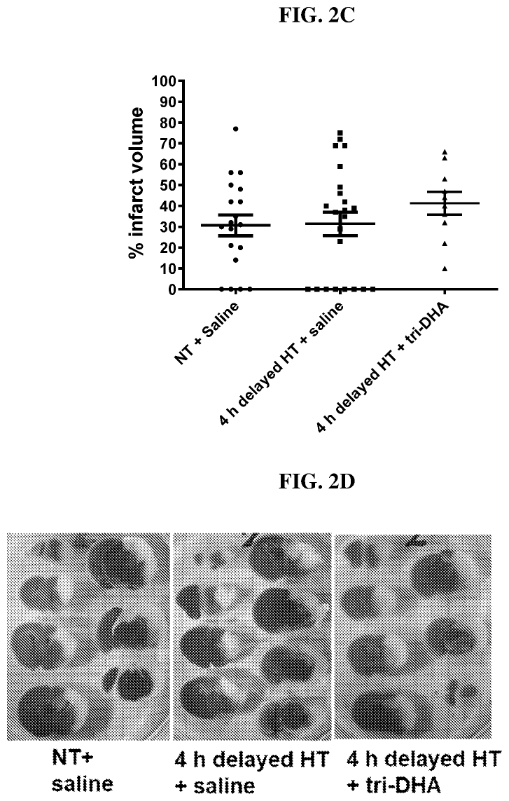

In this example, delayed HT treatment protocols were performed to determine the therapeutic window for neuroprotection after ischemic injury. HT delayed 2 h post-HI showed reduced brain infarct volumes compared to NT animals. Further, HT plus tri-DHA treatment did not offer significant additional protection over that provided by HT alone beginning at 2 h after HI injury although there was a tendency for slightly more reduction in infarct size (% Infarct volume: 31.4±4.1 NT+saline vs 18.8±4.6 HT+saline vs 12.7±4.0 HT+tri-DHA) (FIGS. 2A and 2B). HT treatment delayed at 4 h after HI insult did not offer protection against ischemic injury. Combining HT and tri-DHA treatment with a delay of 4 h after HI injury did not extend the therapeutic window of HT. Although we observed an increase in infarct volume in animals treated with 4 h delayed HT+tri-DHA combination, the difference was not significant compared to NT or HT alone (FIGS. 2C and 2D). Thus, our results indicate that combined treatment of tri-DHA emulsion with HT does not provide additional significant benefit in neuroprotection in ischemic injury.

### DISCUSSION

In this study, the results show that HT administration exerts similar degrees of neuroprotection as that of tri-DHA. Further, combined treatment of HT with tri-DHA emulsion does not confer additional neuroprotection.

Therapeutic HT is a means of neuroprotection well established in the management of acute ischemic brain injuries such as anoxic encephalopathy after cardiac arrest and perinatal asphyxia (28). Randomized trials have shown that HT is also effective in improving neurological outcomes in traumatic brain injury patients (29). Neuroprotective benefits of systemic HT following ischemic stroke have been reported in clinical trials (7). However, the use of HT for acute stroke treatment is still controversial and is limited by logistical challenges (7,30).

HT initiated immediately after HI insult is neuroprotective and the degree of neuroprotection decreases linearly with the delay of initiation of cooling (31,32). In neonatal mouse models of HI injury, HT beginning at 0 or 2 h after HI provides neuroprotection (23), while no studies have assessed the effect of HT when delayed by more than 2 h in mice. The results presented here show that HT is neuroprotective up to 2 h after HI injury and the protection is lost with prolonged 4 h delay in treatment. In a neonatal rat model, Sabir et al. (32) showed that HT delayed up to 6 h after HI insult provides neuroprotection. This may be related to differences in pathways of ischemic injury progression and neuroprotection and/or differences in metabolic rates in mice vs. rats (33). We reported that tri-DHA administered up to 2 h after HI injury significantly reduced (˜50%) cerebral infarct volume, while no protective effect was noted with tri-DHA administration delayed at 4 h after HI injury (20). These present results suggest that HT offers a very similar therapeutic window as tri-DHA treatment. In neonates with HI encephalopathy, selective head cooling with cooling caps or whole body cooling with passive cooling (turning the radiant warmer/incubator off), cool packs and/or commercially available cooling blankets are used for treatment (34,35). A therapeutic window shorter than 6 h is suggested for neonatal HI encephalopathy. (36,37). However, HT initiated at 6 to 24 h after birth may have benefit but there is uncertainty in its effectiveness (38). The basal metabolic rate per kg of body weight is seven times greater in mice than in humans (39) and this may play a major role in providing longer treatment windows for HT in humans in response to HI injury. Therefore, neuroprotection with 2 h delayed treatment in our protocol in mice may translate into longer time windows with HT in humans.

We tested whether DHA might add better neuroprotection as an adjuvant therapy to enhance the efficacy of HT after HI injury. Our results suggest that combining HT and tri-DHA does not enhance neuroprotection or extend the therapeutic window of treatment after HI injury. This is similar to recent findings from a study in a new-born piglet model of HI injury, which showed that combined treatment of HT and DHA had no additional benefits than individual treatments in reducing brain injury and inflammatory markers following HI insult (40). However, another study in a neonatal rat model of HI injury showed that HT plus DHA synergistically reduced brain infarct volume and improved behavioral performances (41). Of interest, the inability to markedly enhance neuroprotection by HT plus tri-DHA treatment is not attributed to a reduction of absorption and distribution of tri-DHA emulsion particles, as demonstrated by our radiolabeled experiments. Further, low uptake of tri-DHA emulsion particles in the brain does not account for the effectiveness of tri-DHA in providing HI neuroprotection. Recent data shows that injected tri-DHA emulsion is taken up by the liver, which is metabolized and secreted to plasma pools of lysophosphatidylcholine and non-esterified fatty acids, facilitating DHA brain transport (22).

Both DHA and HT share common pathways of neuroprotection against HI injury. DHA and HT downregulate pro-apoptotic B-cell lymphoma 2 (BCL-2) associated X (BAX) and upregulate anti-apoptotic BCL-2, resulting in reduced cytochrome c release and decreased caspase activation (17,42). Additionally, DHA and HT promote activation of AKT that stimulate cell proliferation (43,44). Further, it has been reported that in experimental stroke, DHA and HT treatment is capable of decreasing microglial activation and pro-inflammatory cytokines such as interleukin 1β (IL-1β), IL-6 and tumor necrosis factor alpha (TNF-α) (45,46). Additionally, both the treatments inhibit nuclear factor kappa B (NF-κB), a transcription factor that activates many inflammatory signaling pathways (47,48). Further, DHA and HT have also been shown to prevent accumulation or release of excitotoxic amino acids such as glutamate (49,50). Both DHA and HT limit reperfusion-driven acceleration in mitochondrial ROS release and protect against mitochondrial membrane permeabilization (21,51). Thus, we believe that HT and DHA might be acting through similar pathways of neuroprotection, rendering the combination treatment ineffective in further reducing brain injury.

Currently, HT is the only established treatment for moderate to severe encephalopathy in infants (52) and is a promising strategy still under investigation for stroke therapy in adults (53). Successful clinical translation of HT for stroke requires the control of different key parameters of HT therapy including onset time, duration, depth of HT and rewarming speed (11). Although cooling a patient is simple in concept, it is a complex medical procedure that involves coordination of efforts from specially trained health care staff along with preparedness for the management issues that may arise with HT (12,54). Using HT as a treatment for stroke usually requires settings in a tertiary care hospital and is associated with high financial costs (55). Our findings show that HT or injection of tri-DHA emulsion reduce infarct volume and the degree of neuroprotection is similar for both treatments. Omega-3 fatty acids are safe and well tolerated in humans without major adverse effects (56-58). Intravenous injections are a common feasible procedure, which can be easily performed in primary care settings.

### Example 2: Acute Injection of Omega-3 Diglyceride Emulsion Provides Better Protection Against Brain Hypoxic-Ischemic Injury in a Neonatal Mouse Model than Omega-3 Triglyceride Emulsions

Acute treatment with triglyceride lipid emulsions containing both EPA and DHA or DHA alone (tri-DHA) provides neuroprotection after hypoxic-ischemic brain injury by acting within the initial minutes/hours of reperfusion. It is believed that the biological mechanisms affected by tri-DHA and its bioactive mediators, include (i) decreases in generation of mitochondrial reactive oxygen species (ROS); (ii) preservation of mitochondrial functions as demonstrated by maintaining Ca2+ uptake and homeostasis; (iii) blocking free radical production in brain mitochondria within 30 min of reperfusion, and (iv) inhibition of mitochondrial-related apoptotic pathways. The neuroprotection afforded by tri-DHA acute administration was also associated with increased content of DHA in brain mitochondria and of DHA-derived bioactive mediators, such as neuroprotectin D1 (NPD1) and D-series resolvins, in cerebral tissue. Thus, the neuroprotective action afforded by direct administration of NPD1 after hypoxic-ischemic injury was further investigated. We observed that NPD1 treatment induced significant reduction in cerebral infarct volumes compared to the control group, and this was associated with both preserved mitochondrial Ca2+ buffering capacity and reduced mitochondria-related cell death pathways.

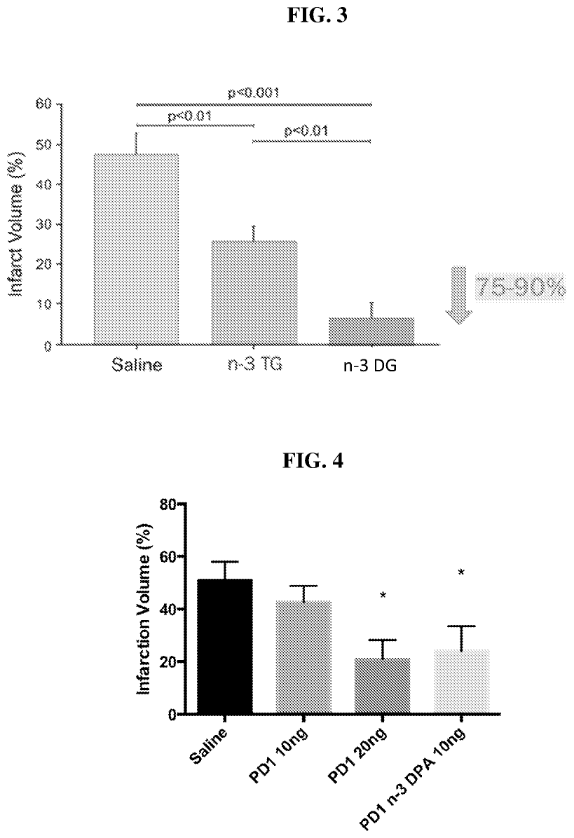

Data further show that neonatal mice treated with omega-3 FAs carried in DG emulsions (containing >90% of total fatty acids as EPA and DHA) exhibited significant reduction in cerebral infarct volumes and omega-3 DG emulsions were far more effective than omega-3 TG emulsions in neonatal HI models. FIG. 3.

We characterized the chemical-physical properties of omega-3 FAs carried in DG emulsions and compared them to TG emulsions. We evaluated the interactions of DG with in vitro models of cellular membranes by NMR spectroscopy. We observed that (i) DG did not adversely affect membrane structures and (ii) incorporated in biological membrane systems more efficiently than TG. To establish if rapid hydrolysis facilitated clearance of diglyceride emulsions, in vitro lipolysis studies were performed by assessing fatty acid release by lipoprotein lipase (LpL)-mediated hydrolysis. We observed that omega-3 DG emulsions had more efficient hydrolysis compared to omega-3 TG. This might explain/contribute to the faster uptake and rapid neuroprotection of DG in ischemic brain injury. These findings highlight the potential of omega-3 FAs delivered in lipid emulsions as strong neuroprotectants in hypoxic-ischemic brain injury, including infants.

In a further study, as shown in FIG. 4, infarct volumes were determined in neonatal mice (10-day old) subjected to hypoxic-ischemic (HI) injury and treated with saline as vehicle or PD1 n-3 DPA at the concentrations of 5, 10 or 20 ng per animal n=15-17. Values are means with SD. Reductions in infarct volume were dose-dependent.

### REFERENCES

1. Wu Y W, et al. Placental pathology and neonatal brain MRI in a randomized trial of erythropoietin for hypoxic-ischemic encephalopathy. Pediatr Res (2020) 87:879-884.
2. Huang L, Zhang L. Neural stem cell therapies and hypoxic-ischemic brain injury. Prog Neurobiol (2019) 173:1-17.
3. Ye X, et al. Purinergic 2X7 receptor/NLRP3 pathway triggers neuronal apoptosis after ischemic stroke in the mouse. Exp Neurol (2017) 292:46-55.
4. Aker K, et al. Thomas N. Therapeutic hypothermia for neonatal hypoxic-ischaemic encephalopathy in India (THIN study): a randomised controlled trial. Arch Dis Child—Fetal Neonatal Ed (2020) 105:405-411.
5. Zhao Y, et al. Pharmacological hypothermia induced neurovascular protection after severe stroke of transient middle cerebral artery occlusion in mice. Exp Neurol (2020) 325:113133.
6. Zgavc T, et al. Experimental and clinical use of therapeutic hypothermia for ischemic stroke: opportunities and limitations. Stroke Res Treat (2011) 2011:689290.
7. Duan Y, et al. New endovascular approach for hypothermia with intrajugular cooling and neuroprotective effect in ischemic stroke. Stroke (2020) 51:628-636.
8. Yenari M A, Han H S. Neuroprotective mechanisms of hypothermia in brain ischaemia. Nat Rev Neurosci (2012) 13:267-278.
9. Jiang J, Yu M, Zhu C. Effect of long-term mild hypothermia therapy in patients with severe traumatic brain injury: 1-year follow-up review of 87 cases. J Neurosurg (2000) 93:546-549.
10. Berger C, et al. Hypothermia and brain-derived neurotrophic factor reduce glutamate synergistically in acute stroke. Exp Neurol (2004) 185:305-312.
11. Wu L, et al. Hypothermic neuroprotection against acute ischemic stroke: The 2019 update. J Cereb Blood Flow Metab (2020) 40:461-481.
12. Lampe J W, Becker L B. State of the art in therapeutic hypothermia. Annu Rev Med (2011) 62:79-93.
13. Kurisu K, et al. Therapeutic hypothermia and neuroprotection in acute neurological disease. Curr Med Chem (2019) 26:5430-5455.
14. Yenari M, et al. Metabolic downregulation. Stroke J Cereb Circ (2008) 39:2910-2917.
15. Cilio M R, Ferriero D M. Synergistic neuroprotective therapies with hypothermia. Semin Fetal Neonatal Med (2010) 15:293-298.
16. Takeyama E, et al. Dietary intake of green nut oil or DHA ameliorates DHA distribution in the brain of a mouse model of dementia accompanied by memory recovery. Nutrients (2019) 11:2371.
17. Mayurasakorn K, et al. Docosahexaenoic acid: brain accretion and roles in neuroprotection after brain hypoxia and ischemia. Curr Opin Clin Nutr Metab Care (2011) 14:158-167.
18. Eady T N, et al. Docosahexaenoic acid complexed to albumin provides neuroprotection after experimental stroke in aged rats. Neurobiol Dis (2014) 62:1-7.
19. Jiang X, et al. A post-stroke therapeutic regimen with omega-3 polyunsaturated fatty acids that promotes white matter integrity and beneficial microglial responses after cerebral ischemia. Transl Stroke Res (2016) 7:548-561.
20. Williams J J, et al. N-3 fatty acid rich triglyceride emulsions are neuroprotective after cerebral hypoxic-ischemic injury in neonatal mice. PLoS ONE (2013) 8:e56233.
21. Mayurasakorn K, et al. DHA but not EPA emulsions preserve neurological and mitochondrial function after brain hypoxia-ischemia in neonatal mice. PloS One (2016) 11:e0160870.
22. Manual Kollareth D J, et al. Acute injection of a DHA triglyceride emulsion after hypoxic-ischemic brain injury in mice increases both DHA and EPA levels in blood and brain. Prostaglandins Leukot Essent Fatty Acids (2020) 162:102176.
23. Reinboth B S, et al. Endogenous hypothermic response to hypoxia reduces brain injury: Implications for modeling hypoxic-ischemic encephalopathy and therapeutic hypothermia in neonatal mice. Exp Neurol (2016) 283:264-275.
24. Galinsky R, et al. In the era of therapeutic hypothermia, how well do studies of perinatal neuroprotection control temperature? Dev Neurosci (2017) 39:7-22.
25. Qi K, et al. Effects of particle size on blood clearance and tissue uptake of lipid emulsions with different triglyceride compositions. J Parenter Enter Nutr (2003) 27:58-64.
26. Manual Kollareth D J, et al. Radiolabeled cholesteryl ethers: A need to analyze for biological stability before use. Biochem Biophys Rep (2018) 13:1-6.
27. Zirpoli H, et al. NPD1 rapidly targets mitochondria-mediated apoptosis after acute injection protecting brain against ischemic injury. Exp Neurol (2020)113495.
28. van der Worp H B, et al. Therapeutic hypothermia for acute ischemic stroke: ready to start large randomized trials? J Cereb Blood Flow Metab (2010) 30:1079-1093.
29. Dietrich W D, Bramlett H M. Therapeutic hypothermia and targeted temperature management for traumatic brain injury: Experimental and clinical experience. Brain Circ (2017) 3:186-198.
30. Zhao H, Steinberg G. Limited therapeutic time windows of mild-to-moderate hypothermia in a focal ischemia model in rat. Stroke Res Treat (2011) 2011: doi: 10.4061/2011/131834
31. Gunn A J. Cerebral hypothermia for prevention of brain injury following perinatal asphyxia. Curr Opin Pediatr (2000) 12:111-115.
32. Sabir H, et al Immediate hypothermia is not neuroprotective after severe hypoxia-ischemia and is deleterious when delayed by 12 hours in neonatal rats. Stroke (2012) 43:3364-3370.
33. Du Y, et al. Differential subnetwork of chemokines/cytokines in human, mouse, and rat brain cells after oxygen-glucose deprivation. J Cereb Blood Flow Metab Off J Int Soc Cereb Blood Flow Metab (2017) 37:1425-1434.
34. Akula V P, et al. A randomized clinical trial of therapeutic hypothermia mode during transport for neonatal encephalopathy. J Pediatr (2015) 166:856-861.e2.
35. Peliowski-Davidovich A. Hypothermia for newborns with hypoxic ischemic encephalopathy. Paediatr Child Health (2012) 17:41-43.
36. Kendall G S, et al. Cooling on retrieval study group. Passive cooling for initiation of therapeutic hypothermia in neonatal encephalopathy. Arch Dis Child Fetal Neonatal Ed (2010) 95:F408-412.
37. Gunn A J, Groenendaal F. Delayed neuroprotection in the era of hypothermia: What can we add? J Clin Neonatol (2016) 5:3.
38. Laptook A R, et al. Effect of therapeutic hypothermia initiated after 6 hours of age on death or disability among newborns with hypoxic-ischemic encephalopathy. JAMA (2017) 318:1550-1560.
39. Demetrius L. Of mice and men. EMBO Rep (2005) 6:S39-S44.
40. Huun M U, et al. DHA and therapeutic hypothermia in a short-term follow-up piglet model of hypoxia-ischemia: Effects on H+MRS biomarkers. PloS One (2018) 13:e0201895.
41. Berman D R, et al. Docosahexaenoic acid augments hypothermic neuroprotection in a neonatal rat asphyxia model. Neonatology (2013) 104:71-78.
42. An H, et al. Phenothiazines enhance mild hypothermia-induced neuroprotection via PI3K/Akt regulation in experimental stroke. Sci Rep (2017) 7:7469.
43. Eady T N, et al. Docosahexaenoic acid signaling modulates cell survival in experimental ischemic stroke penumbra and initiates long-term repair in young and aged rats. PloS One (2012) 7:e46151.
44. Zhao H, et al. Akt contributes to neuroprotection by hypothermia against cerebral ischemia in rats. J Neurosci Off J Soc Neurosci (2005) 25:9794-9806.
45. Cai W, et al. Post-stroke DHA Treatment Protects Against Acute Ischemic Brain Injury by Skewing Macrophage Polarity Toward the M2 Phenotype. Transl Stroke Res (2018) 9:669-680.
46. Lee J H, et al. Regulation of therapeutic hypothermia on inflammatory cytokines, microglia polarization, migration and functional recovery after ischemic stroke in mice. Neurobiol Dis (2016) 96:248-260.
47. Zhang W, et al. Omega-3 polyunsaturated fatty acid supplementation confers long-term neuroprotection against neonatal hypoxic-ischemic brain injury through anti-inflammatory actions. Stroke (2010) 41:2341-2347.
48. Yenari M A, Han H S. Influence of hypothermia on post-ischemic inflammation: role of nuclear factor kappa B (NFkappaB). Neurochem Int (2006) 49:164-169.
49. Moreira J D, et al. Dietary omega-3 fatty acids attenuate cellular damage after a hippocampal ischemic insult in adult rats. J Nutr Biochem (2010) 21:351-356.
50. Zhang H, et al. Therapeutic effect of post-ischemic hypothermia duration on cerebral ischemic injury. Neurol Res (2008) 30:332-336.
51. Gong P, et al. Hypothermia-induced neuroprotection is associated with reduced mitochondrial membrane permeability in a swine model of cardiac arrest. J Cereb Blood Flow Metab Off J Int Soc Cereb Blood Flow Metab (2013) 33:928-934.
52. Oorschot D E, et al. Treatment of neonatal hypoxic-ischemic encephalopathy with erythropoietin alone, and erythropoietin combined with hypothermia: history, current status, and future research. Int J Mol Sci (2020) 21:1487.
53. Chen J, et al. Endovascular hypothermia in acute ischemic stroke: pilot study of selective intra-arterial cold saline infusion. Stroke (2016) 47:1933-1935.
54. Li L, et al. Combination treatment with methylene blue and hypothermia in global cerebral ischemia. Mol Neurobiol (2018) 55:2042-2055.
55. Kim J J, et al. Cost-effective therapeutic hypothermia treatment device for hypoxic is chemic encephalopathy. Med Devices (2013) 6:1-10.
56. Zhang M-M, et al. The efficacy and safety of omega-3 fatty acids on depressive symptoms in perinatal women: a meta-analysis of randomized placebo-controlled trials. Transl Psychiatry (2020) 10: doi:10.1038/s41398-020-00886-3.
57. Defilippis A P, et al. Omega-3 Fatty acids for cardiovascular disease prevention. Curr Treat Options Cardiovasc Med (2010) 12:365-380.
58. Calder P C, Deckelbaum R J. Intravenous fish oil in hospitalized adult patients: reviewing the reviews. Curr Opin Clin Nutr Metab Care (2013) 16:119-123.

TABLE 1
Rectal temperature measurements immediately after hypoxia
(0 h) and at 1, 2, 3 and 4 h during hypothermia (HT) in
neonatal mice subjected to hypoxic-ischemic (HI) injury

| | HT | HT + tri-DHA |
|---|---|---|
| End of hypoxia (0 h) | 35.2 ± 0.48 | 35.6 ± 0.41 |
| 1 h after HI | 32.0 ± 0.16 | 32.2 ± 0.25 |
| 2 h after HI | 31.4 ± 0.24 | 32.0 ± 0.17 |
| 3 h after HI | 31.0 ± 0.19 | 32.1 ± 0.15 |
| 4 h after HI | 31.6 ± 0.26 | 31.8 ± 0.28 |

Temperatures (° C.) are expressed as mean ± SEM. n = 7-9

1. A method for protecting against brain injury associated with neonatal Hypoxic-Ischemic Encephalopathy (HIE), the method comprising: administering to a subject in need, an omega-3 fatty acid (FA) diglyceride (DG) emulsion and/or and omega-3 FA triglyceride (TG) emulsion.

2. The method of claim 1, wherein the subject is a prenatal subject at risk of HIE, and the DG and/or TG emulsion is administered to the pregnant mother.

3. The method of claim 1, wherein the subject is intrapartum, and the DG or TG emulsion is administered intravenously or by nasogastric tube to the mother prior to the onset of labor or after the onset of labor.

4. The method of claim 2 or 3, wherein the emulsion is administered intravenously as one or more bolus injections or by continuous infusion, or as a combination of one or more bolus loading doses followed by infusion of one or more additional intravenous doses.

5. The method of claim 1, wherein the subject is a newborn, and the DG and/or TG emulsion is first administered to the newborn within about twelve hours of delivery.

6. The method of claim 5, wherein the newborn is preterm or a term neonate.

7. The method of claim 5 or 6, wherein at least a first dose of the emulsion is administered within about six hours of delivery, or within about four hours of delivery, within about two hours of delivery.

8. The method of any one of claims 5 to 7, wherein the emulsion is administered via a nasogastric (NG) tube or intravenously, either as one or more bolus injections and/or as a continuous infusion.

9. The method of any one of claims 1 to 8, wherein the newborn is treated with therapeutic hypothermia (HT).

10. The method of claim 9, wherein the emulsion is first administered before, during, or after HT.

11. The method of claim 9 or 10, wherein the HT is initiated within about twelve hours of delivery.

12. The method of claim 11, wherein the HT is initiated within about ten hours of delivery.

13. The method of claim 11, wherein the HT is initiated within about eight hours of delivery.

14. The method of claim 11, wherein the HT is initiated within about six hours of delivery.

15. The method of claim 11, wherein the HT is initiated after about six hours of delivery, or after about eight hours of delivery, or after about ten hours of delivery, or after about twelve hours of delivery.

16. The method of any one of claims 1 to 15, wherein omega-3 FA TG emulsion is administered.

17. The method of any one of claims 1 to 15, wherein omega-3 FA DG emulsion is administered.

18. The method of claim 16 or 17, wherein the FAs comprise at least about 50% eicosapentaenoic acid (EPA) and/or docosahexaenoic acid (DHA) by weight of the fatty acids.

19. The method of claim 18, wherein the fatty acids comprise at least about 60% EPA and/or DHA by weight.

20. The method of claim 18, wherein the fatty acids comprise at least about 70% EPA and/or DHA by weight.

21. The method of any one of claims 18 to 20, wherein the fatty acids comprise both EPA and DHA.

22. The method of claim 20, wherein the ratio of EPA:DHA is about 4:1 to about 1:4.

23. The method of claim 22, wherein the ratio of EPA:DHA is about 2:1 to about 1:2, and is optionally about 1:1.

24. The method of any one of claims 16 to 23, wherein the FAs further comprise docosapentaenoic acid (DPA).

25. The method of any one of claims 15 to 24, wherein the FAs further comprise arachidonic acid (ARA).

26. The method of claim 25, wherein the FAs comprise about 1 to about 40% ARA by weight.

27. The method of any one of claims 1 to 25, wherein the DG or TG, or emulsions thereof, further comprise one or more specialized pro-resolving mediators (SPMs).

28. The method of claim 27, wherein the SPM(s) comprise one or more lipoxins, (neuro)protectins, resolvins, and maresins.

29. The method of any one of claims 15 to 28, wherein the emulsions further comprise medium chain fatty acids (MCFAs), either as free FAs or esterified as TG and/or DG.

30. The method of claim 29, wherein the MCFAs are about 1% to about 20% of the fatty acids in the TG and/or DG emulsion.

31. The method of any one of claims 1 to 30, wherein the emulsion comprises about 10% to about 30% TG and/or DG by weight of the composition.

32. The method of claim 31, wherein the emulsion comprises about 15% to about 25% DG by weight of the composition.

33. The method of any one of claims 1 to 32, wherein the emulsions have a mean particle size of 200 nm or less.

34. The method of claim 33, wherein the emulsions have a zeta potential of about −40 mV or more negative than about −40 mV.

35. The method of any one of claims 1 to 34, wherein the subject receives a single administration of the emulsion.

36. The method of any one of claims 1 to 35, wherein the subject receives at least two and up to six administrations of the emulsion.

37. The method of claim 36, wherein the emulsions are administered no more frequently than about once every 12 hours.

38. The method of claim 36, wherein emulsion is administered over the course of about one day to about one week.

39. The method of claim 36, wherein emulsion is administered about daily.

40. The method of any one of claims 1 to 39, wherein each administration is a dose of about 0.05 to about 5 g of the emulsified DG or TG per kg body weight.

41. The method of claim 40, wherein each administration is about 0.5 to about 4 g of the emulsified DG or TG per kg body weight.

42. A method for protecting against brain injury associated with Hypoxic-Ischemic Encephalopathy (HIE) in a new born, the method comprising: administering to a pregnant mother carrying a child at risk of HIE, either prenatal or intrapartum, an intravenous injection of omega-3 FAs DG or TG emulsion; and optionally treating the newborn after delivery with a therapeutic hypothermia (HT) regimen and/or omega-3 FA DG or TG emulsion therapy.

43. The method of claim 42, wherein the DG or TG emulsion is administered intravenously to the mother after the onset of labor.

44. The method of claim 42 or claim 43, wherein the birth is term or preterm.

45. The method of any one of claims 42 to 44, wherein the emulsion is administered intravenously as a bolus or by continuous infusion, or as a combination of one or more bolus loading doses followed by infusion of one or more additional doses.

46. The method of any one of claims 42 to 45, wherein n-3 FA DG emulsion is administered to the newborn within about twelve hours of delivery.

47. The method of claim 46, wherein the n-3 FA DG emulsion is administered within about six hours of delivery, or within about four hours of delivery, within about two hours of delivery.

48. The method of claim 46 or 47, wherein the n-3 FA DG or TG emulsion is administered to the new born via a NG tube or intravenously, either as a bolus and/or as a continuous infusion.

49. The method of any one of claims 42 to 48, wherein the new born is treated with therapeutic hypothermia (HT).

50. The method of claim 49, wherein the HT is initiated within about twelve hours of delivery.

51. The method of claim 49, wherein the HT is initiated within about ten hours of delivery.

52. The method of claim 49, wherein the HT is initiated within about eight hours of delivery.

53. The method of claim 49, wherein the HT is initiated within about six hours of delivery.

54. The method of claim 49, wherein the HT is initiated after about six hours of delivery, or after about eight hours of delivery, or after about ten hours of delivery, or after about twelve hours of delivery.

55. The method of any one of claims 42 to 54, wherein the fatty acids comprise at least about 50% EPA and/or DHA by weight.

56. The method of claim 55, wherein the fatty acids comprise at least about 60% EPA and/or DHA by weight.

57. The method of claim 55, wherein the fatty acids comprise at least about 70% EPA and/or DHA by weight.

58. The method of any one of claims 55 to 57, wherein the fatty acids comprise both EPA and DHA.

59. The method of claim 58, wherein the ratio of EPA:DHA is about 4:1 to about 1:4.

60. The method of claim 59, wherein the ratio of EPA:DHA is about 2:1 to about 1:2, and is optionally about 1:1.

61. The method of any one of claims 55 to 60, wherein the fatty acids further comprise DPA.

62. The method of any one of claims 55 to 61, wherein the fatty acids further comprise ARA.

63. The method of claim 62, wherein the fatty acids comprise from about 1% to about 40% ARA by weight.

64. The method of any one of claims 42 to 63, wherein the DG or TG, or emulsions thereof, further comprise one or more SPMs.

65. The method of claim 64, wherein the SPM(s) comprise one or more lipoxins, (neuro)protectins, resolvins, and maresins.

66. The method of any one of claims 42 to 65, wherein the emulsion further comprises medium chain fatty acids (MCFA), either as free fatty acids or esterified as diglycerides.

67. The method of claim 66, wherein the MCFA is from about 1% to about 20% by weight of the fatty acids in the emulsion.

68. The method of any one of claims 42 to 67, wherein the emulsion comprises about 10% to about 20% DG oil by weight of the composition.

69. The method of claim 68, wherein the emulsion comprises about 15% to about 25% DG or TG oil by weight of the composition.

70. The method of any one of claims 42 to 69, wherein the emulsions have a mean particle size of 200 nm or less.

71. The method of claim 70, wherein the emulsions have a zeta potential of about −40 mV or more negative than about −40 mV.

72. The method of any one of claims 42 to 71, wherein the neonate receives a single bolus injection or continuous infusion of the emulsion.

73. The method of any one of claims 42 to 71, wherein the neonate receives at least two and up to six bolus injections or infusions of the emulsion.

74. The method of claim 73, wherein the bolus injections or infusions are administered no more frequently than about once every 12 hours.

75. The method of claim 73, wherein injections are administered over the course of about one day to about one week.

76. The method of claim 75, wherein the injections are administered about daily.

77. The method of any one of claims 42 to 76, wherein the bolus administration is from about 0.05 to about 5 g of the emulsified DG or TG per kg body weight.

78. The method of claim 77, wherein the bolus administration is about 0.5 g to about 4 g of the emulsified DG or TG per kg body weight.

79. A method for protecting against brain injury associated with Hypoxic-Ischemic Encephalopathy (HIE), the method comprising: administering to a neonatal subject in need, omega-3 FAs DG or TG emulsion within about twelve hours of delivery, and treating the subject with a therapeutic hypothermia (HT) regimen.

80. The method of claim 79, wherein the neonatal subject is first treated with the emulsion before, during, or after HT.

81. The method of claim 79 or 80, wherein the neonatal subject is a preterm or term neonate.

82. The method of any one of claims 79 to 81, wherein at least a first dose of DG or TG emulsion is administered within about six hours of delivery, or within about four hours of delivery, within about two hours of delivery.

83. The method of any one of claims 79 to 82, wherein the DG or TG emulsion is administered via a NG tube or intravenously, either as a bolus and/or as a continuous infusion.

84. The method of any one of claims 79 to 83, wherein the HT is initiated within about twelve hours of delivery.

85. The method of claim 84, wherein a dose of the DG or TG emulsion is delivered within about four hours of delivery, and the HT is initiated within about ten hours of delivery.

86. The method of claim 84, wherein a dose of the DG or TG emulsion is delivered within about four hours of delivery, and the HT is initiated within about eight hours of delivery.

87. The method of claim 84, wherein a dose of the DG emulsion is delivered within about four hours of delivery, and the HT is initiated within about six hours of delivery.

88. The method of claim 84, wherein a dose of the DG emulsion is delivered within about two hours of delivery, and the HT is initiated after about six hours of delivery, or after about eight hours of delivery, or after about ten hours of delivery, or after about twelve hours of delivery.

89. The method of any one of claims 80 to 88, wherein the DG or TG emulsion is administered intravenously as a bolus or by continuous infusion, or as a combination of a bolus loading dose followed by infusion of one or more additional doses.

90. The method of any one of claims 80 to 89, wherein the fatty acids comprise at least about 50% EPA and/or DHA by weight.

91. The method of claim 90, wherein the fatty acids comprise at least about 60% EPA and/or DHA by weight.

92. The method of claim 91, wherein the fatty acids comprise at least about 70% EPA and/or DHA by weight.

93. The method of any one of claims 90 to 92, wherein the fatty acids comprise both EPA and DHA.

94. The method of claim 93, wherein the ratio of EPA:DHA is from about 4:1 to about 1:4.

95. The method of claim 94, wherein the ratio of EPA:DHA is from about 2:1 to about 1:2, and is optionally about 1:1.

96. The method of any one of claims 90 to 95, wherein the fatty acids further comprise DPA.

97. The method of any one of claims 90 to 96, wherein the fatty acids further comprise ARA.

98. The method of claim 97, wherein the fatty acids comprise about 1% to about 40% ARA by weight.

99. The method of any one of claims 90 to 98, wherein the DG or TG, or emulsions thereof, further comprise one or more SPMs.

100. The method of claim 99, wherein the SPM(s) comprise one or more lipoxins, (neuro)protectins, resolvins, and maresins.

101. The method of any one of claims 90 to 100, wherein the emulsion further comprises MCFAs, either as free FAs or esterified as DG or TG.

102. The method of claim 101, wherein the MCFAs are about 1% to about 20% by weight of the fatty acids in the DG or TG emulsion.

103. The method of any one of claims 79 to 102, wherein the emulsions comprise about 10% to about 30% DG or TG oil by weight.

104. The method of claim 103, wherein the emulsion comprises about 15% to about 25% DG or TG oil by weight.

105. The method of any one of claims 79 to 104, wherein the emulsions have a mean particle size of 200 nm or less.

106. The method of claim 105, wherein the emulsions have a zeta potential of about −40 mV or more negative than about −40 mV.

107. The method of any one of claims 79 to 106, wherein the subject receives a single bolus injection or infusion of the emulsion.

108. The method of any one of claims 79 to 106, wherein the subject receives at least two and up to six bolus injections or infusions of the emulsion.

109. The method of claim 108, wherein bolus injections or infusions are administered no more frequently than about once every twelve hours.

110. The method of claim 108, wherein bolus injections or infusions are administered over the course of about one day to about one week.

111. The method of claim 108, wherein the bolus injections or infusions are administered about daily.

112. The method of any one of claims 79 to 111, wherein a single administration comprises about 0.05 to about 5 g of the emulsified DG or TG per kg body weight.

113. The method of claim 112, wherein a single administration comprises about 2 g to about 4 g of the emulsified DG or TG per kg body weight.

114. A pharmaceutical composition comprising an effective amount of omega-3 DG or TG oil emulsified with one or more emulsifiers, the DG or TG oil comprising esterified FAs that are at least about 50% EPA and DHA by weight, and from 1% to about 30% ARA by weight.

115. The pharmaceutical composition of claim 114, wherein the FAs comprise at least about 60% EPA and DHA by weight.

116. The pharmaceutical composition of claim 114, wherein the FAs comprise at least about 70% EPA and DHA by weight.

117. The pharmaceutical composition of any one of claims 114 to 116, wherein the ratio of EPA:DHA is about 4:1 to about 1:4.

118. The pharmaceutical composition of claim 117, wherein the ratio of EPA:DHA is about 2:1 to about 1:2, and is optionally about 1:1.

119. The pharmaceutical composition of any one of claims 114 to 118, wherein the FAs further comprise DPA.

120. The pharmaceutical composition of any one of claims 114 to 119, wherein the DG or TG, or emulsions thereof, further comprise one or more SPMs.

121. The method of claim 120, wherein the SPM(s) comprise one or more lipoxins, (neuro)protectins, resolvins, and maresins.

122. The pharmaceutical composition of any one of claims 114 to 121, wherein the emulsions further comprise MCFAs, either as free FAs or esterified in DG or TG.

123. The pharmaceutical composition of claim 122, wherein the MCFAs are about 1% to about 20% of the FAs by weight in the emulsion.

124. The pharmaceutical composition of any one of claims 114 to 123, wherein the emulsion comprises about 10% to about 30% DG or TG oil by weight.

125. The pharmaceutical composition of claim 124, wherein the emulsion comprises about 15% to about 25% DG or TG oil by weight.

126. The pharmaceutical composition of any one of claims 114 to 125, wherein the emulsions have a mean particle size of 200 nm or less.

127. The pharmaceutical composition of claim 126, wherein the emulsions have a zeta potential of about −40 mV or more negative than about −40 mV.

128. The pharmaceutical composition of any one of claims 114 to 127, wherein the emulsifiers include one or more of phospholipid emulsifiers, phosphoglyceride emulsifiers, and medium and/or long chain FA emulsifiers.

129. The pharmaceutical composition of claim 128, wherein the emulsifiers include at least one selected from phosphatidylcholine, phosphatidylethanolamine, phosphatidylinositol, phosphatidylserine, and phosphatidic acid.

130. The pharmaceutical composition of claim 128, wherein the emulsifiers further comprise one or more of medium chain or long chain FAs.

131. The pharmaceutical composition of claim 130, wherein the emulsifiers comprise a saturated FA, optionally selected from lauric acid, myristic acid, palmitic acid, and stearic acid; and/or comprise an unsaturated FA, optionally selected from oleic acid or linolenic acid.

132. The pharmaceutical composition of claim 131, wherein the emulsifiers comprise phosphatidylcholine and sodium oleate.

133. The pharmaceutical composition of any one of claims 114 to 132, wherein the composition is approximately isotonic with human blood, and optionally comprises one or more polyols, such as glycerol, sorbitol, xylitol, and/or glucose.

134. The pharmaceutical composition of any one of claims 114 to 133, comprising one or more antioxidants, such as one or more of α-tocopherol, β-tocopherol, γ-tocopherol, and an ascorbyl ester.

135. The pharmaceutical composition of any one of claims 114 to 134, further comprising a metal chelating agent, which is optionally EDTA or EGTA.

136. The pharmaceutical composition of claim 135, consisting essentially of DG oil, water, glycerol, EDTA or EGTA, phosphatidylcholine, and sodium oleate.
